(19) United States
(12) Patent Application Publication
(10) Pub. No.: US 2011/0172684 A1
(43) Pub. Date: Jul. 14, 2011

(54) PROSTHESIS FOR ANASTOMOSIS

(76) Inventor: Luiz Gonzaga Granja Filho, Recife/PE (BR)

(21) Appl. No.: 12/303,651

(22) PCT Filed: Jun. 6, 2007
(86) PCT No.: PCT/BR07/00149

§ 371 Date: Mar. 16, 2011

(30) Foreign Application Priority Data
Jun. 6, 2006 (BR) PI 0602381-9

(51) Int. Cl. A61B 17/11

(52) U.S. Cl. 606/153

(57) ABSTRACT

Prosthetic devices are provided that are used for end-to-side, end-to-end and side-to-side anastomosis, without clamping and sutureless or with quick clamping and sutureless, where the tubular member of the prosthesis, besides having its intraluminal part angled in relation to the flange, can also have more than one intraluminal part, in form of a “Y”, a “T”, and other variations. The flange can also be with or without openings or small handles and chamfers, allowing the tie-up to the external part of the tissue, vein, artery or tubular organ to eliminate the contact of foreign bodies inside the anastomosis. The prosthesis can also be without flange or have a tubular member with tapered center to peripheral part or from external part to intraluminal one, and it can be cut in the desired size and gauge. Therefore, the prosthesis can have varied sizes and shapes to accommodate varied graft sizes and types in a simultaneous way.

### FIELD OF INVENTION

The present invention generally refers to anastomotic devices and, more specifically, to a prosthetic device with angled intraluminal parts, articulable or fixed, single or multiples, that allows anastomosis without clamping and sutureless, where at least one vascular graft, or any other graft, is inserted in the lumen of the prosthesis and reversed by jacketing to cover part of the prosthesis that will remain inside the graft (vein, artery or tissue), and it is fixed in the tubular member prosthesis by round point. The prosthesis flange can have a plurality of spaced openings on the peripheral part or chamfers in the perimeter, allowing the prosthesis to be sutured in the tissue, vein, artery or any other organ out of anastomosis, eliminating the main causes of obstruction of anastomosis, which is the insertion of foreign bodies inside its lumen.

### DESCRIPTION OF THE PRIOR ART

A prior art presents several trials provide solutions for anastomotic devices projected to correct vascular abnormalities, which present the following typical features:

The North-American Patent U.S. No. No. 3,254,650, of Jun. 7, 1966, describes a method and devices to execute anastomosis procedures by applying with adhesive two separated connectors in a body member and removing this body member portion contained among the connectors, joining the said connection devices for joining the remaining portions of the body member.

The U.S. Pat. No. 3,265,069, of Aug. 9, 1966, describes devices or instruments for use by surgeons in reunion of body ducts, which in the course of operations were separated. The instruments comprise a pair of elongated similar elements and articulatedly connected, in an intermediary manner, and with an support for finger retention in a distal end, comprising a generally cylindrical shape with a cylindrical channel that passes through it in the other distal end, in order to receive tubular body ducts kept by the instrument while the body ducts are reconnected.

U.S. Pat. No. 3,774,615, of Nov. 27, 1973, describes a device to connect the end of interrupted tubular organs without sewing, comprising a connecting ring on which the end of the interrupted organ are pulled, the ring is preferably locked up by a fixation resource. The ring and fixation resource are made of inert material, and preferably a hydrophile gel that can be dilated until its equilibrium or can be a hydrogel incompletely dilated, which is submitted to additional dilatation where it is applied. The connecting ring can be supplied with a groove and can be placed in a ring shaped fixation resource and kept there joining it to the fixation resource in the groove or simply kept by a screw. Two connection rings can also be used and kept joined by a coupling member.

The document U.S. Pat. No. 4,366,819, of Jan. 4, 1983, describes an anastomotic joint for surgery with a graft of coronary artery deviance comprising a mounting of four elements including a cylindrical tube with at least one locking indentation of ring flange in one influx end and a plurality of grooves of locking ring in a flow end; a ring flange with a central opening and a plurality of long and short spigots, the long spigots are engaged in the locking indentation, with a graft engaged among them; a fixation ring with a central opening and a plurality of spigots positioned around the opening; and a locking ring with a opening with a plurality of locking ring edges for engaging with the locking ring grooves. In surgical implants, an aortic wall with a hole engages between the ring flange and the fixation ring and is kept in this position by spigots of the fixation ring, and the four elements engage together forming an integral anastomotic joint. A first alternate modality includes an anastomotic joint of three elements with a combination of fixation ring and locking ring. A second alternate modality includes an anastomotic joint of four elements with a slightly jolted end in a influx end, exposing the graft material in the anastomotic “ostium”.

Other prior arts are equally mentioned, base don some information of “The Cardiothoracic Surgery Network”. The “Simmetry Aortic Connector System”, developed by St. Jude Medical, is a connector made with nitinol, selected by vein diameter with an adventitia removed to allow adjust of the connector and to prevent its displacement by the blood current. Then, the device may make an angle of 90° with the aorta. Among the disadvantages, there is the fact that it can be used only in extreme cases due to the difficult usage of this technique; it did not obtain a satisfactory result in many surgeries and it is being drowned out of market by the manufacturer; it is not applicable in calcified aorta; presents suture; presents contact with blood flow (foreign body); it does not widen the anastomosis area (restrictive anastomosis); performs only one anastomosis at a time; it is a product restrict to end-to-side anastomosis; a great mobilization of the venous graft occurs, damaging it, and can eventually form thrombus; there is a risk of perforation of the posterior wall of aorta; and the adventitia is removed (most resistant vascular layer).

Other known device is the PAS-Port™System, a device used in 3 steps, and the vein wall is mounted over the device and is manually reversed on it, by tool and adapted to aorta with a angle of 90° . The method alerts that the surgeon shall select with due care the point of aorta and the vein size. The device is made of stainless steel and is available in only one size that allows the use of veins with external diameter of 4 to 6 mm, aorta with an internal diameter of 18 mm. It is available in only one size, limiting its applicability. As disadvantages of this prior art, the device has contact with blood flow (foreign body); it does not widen the anastomosis area (restrictive anastomosis); it uses veins with external diameter of 4 to 6 mm and aorta with an internal diameter of 18 mm; it does not perform multiple nor visceral anastomosis; it performs just only end-to-side anastomosis; a great mobilization of the used biological graft occurs, damaging its inner layer, which generates the formation of thrombus; there is a big risk of kinking at the origin (angle of 90°) and risk of posterior wall perforation in the aorta at the moment the device is introduced under its light; the suture is substituted with disadvantages by stainless steel (9 pins, distant among them, maximizing the risk of bleeding).

Also as prior art, there is the CorLink Device, currently commercialized by Ethicon/Johnson & Johnson, that allows the creation of anastomosis between the ascending aorta and a saphenous vein segment. Aortic Anastomotic Device (AAD) is a self-expanded device with extra luminal nitinol constituted by a de um central cylinder with five interconnected elliptical arches and 2 groups of 5 pins in the end portion of the cylinder. The pins, after the eversion of venous walls in the device, fix the aggregate penetrating into the venous graft wall. A blade makes an opening in the wall of aorta and permits the coupling of AAD, which also fix the wall of aorta by pins. With this device: it poses a serious risk of bleeding, especially in friable aortas, thin, calcified or fibrous, restricting its applicability, also with risks, even in aortas with normal walls; in small gauge anastomosis, there is a risk of thrombosis, hyperplasia, intimal proliferation and fibrosis (reaction to foreign body type in origin of anastomosis) with consequent stenosis resulting in occlusion of anastomosis; sutures are used in some cases; there is cases of infarction caused by equipment; there is a recurring need of re-operations in patients; the device presents contact with blood flow (foreign body); it is not flexible; it does not multiple anastomosis; an inadequate mobilization of venous graft occurs, and can cause damage to its intimal layer, it could form thrombus; it is used only in extreme cases because it is a technique of complex usage; the suture is substituted by stainless steel in contact with blood flow.

Another known device is the St Jude Distal Connector that consists of a stainless steel clip mounted on a catheter, comprising a balloon for subsequent expansion and connector mounting. The catheter is introduced backward from the end, by doing a small hole in the anastomosis site, the clip fixes the vein in the hole, the catheter goes to coronary and releases the connector. The catheter is removed and a suture is done in side-to-side anastomosis. With St Jude Distal Connector, occurrence of leakage problems were detected in 20% of the used connectors; the use of a metallic clip requires due care for handling to avoid distortion in the anastomosis; late angiographies reveal smaller circular diameter of anastomosis made with o St. Jude Distal Connector, when compared to controls made with conventional suture; there is remarkably risk of bleeding and the graft is very mobilized, and lacerations can occur in its inner layer, allowing the formation of thrombus.

The HeartFlo™ is a multi-suture instrument for anastomosis with wires automatically applied in end-to-side and side-to-side anastomosis. The surgeon manually ties the suture wires (10 wires) and concludes the anastomosis similarly to the traditional process. Besides of being a product of complex handling, it makes suture in anastomosis (keeping the undesirable foreign body in the internal origin of the anastomosis) and is restricted to end-to-side and side-to-side anastomosis. There is also an excessive mobilization of graft, and can cause lesions in its intimal layer, which would be the inductor that forms the thrombus.

Another technique and known device is the Solem Graft connector, produced by the Swedish company Jomed. It is constituted by a stent made of nickel and titanium coated with polytetrafluorethylen used to connect the internal thoracic artery the left anterior descending coronary artery. The results has not been satisfactory, because it poses risk of bleeding; there is also an excessive mobilization of graft, probably damaging intimal layers, allowing the formation of thrombus; it is not flexible, by this fact, causes trauma to grafts; it does not make multiple anastomosis, at a single time; presents contact with blood flow (foreign body); and is frequent the need of-operations.

The Magnetic Vascular Positioner System is produced by Ventrica and comprises 4 magnetic rings and the anastomosis is processed by magnetic attraction of 4 ports. However, initial experimental results demonstrate leakage, also a undesired contact of materials with blood flow. On the other hand, it is necessary to be careful to avoid the capitation of excess of tissue among the magnets. With this system, there is also a need of suture in some cases; there is occurrence of infarction caused by equipment; and is frequent the need of-operations in patients; and also requires clamping.

Also, as a device known by the medical area, the Combined Anastomotic Device and Tissue Adhesive, developed by Grundeman & Borst group, combines micro mechanical technique with use of adhesive (glue). The use of this method can result in leakages and need traditional sutures; it is frequent the need of re-operation due to leakage/bleeding; and performs only one anastomosis at a time.

Finally, it is also experimentally practiced anastomosis assisted by laser, where the results are not different from conventional isolated sutures, because there is a need of suture in some cases; there is a risk of bleeding e leakage; and does not perform multiple anastomosis.

Even so divulged nowadays, anastomosis with damper, by insecurity, and almost totality of surgeons perform conventional sutures throughout the route of anastomosis, with an intention of avoiding leakages and bleedings, it means the use of clampers just makes the procedure more expensive, once the conventional suture is also applied.

In short, the conventional anastomosis, with clamping and with suture, standardized in 1906 by Alexis Carrel, remains the first choice for any type of anastomosis and organs to be anastomosed.

With an expectation of changing the current situation, the Brazilian patent no. PI 9706197-2, describes and claims a prosthesis for vascular anastomosis, or in any other organ or tissue, without the use of clamping and sutureless, solving, in an elegant and efficient manner, the limitations inherent to prosthesis of the above mentioned prior art, when used in vascular anastomosis performed, mainly in thin aortas, calcified and friable; or in any other application where a clamping of a vein or artery can pose excessive trauma for conditions of a given patient. The prosthesis that is subject of that request allows the embodiment of fast and safe anastomosis, without obstruction of vein or artery lumen of which anastomosis is made, also allows anastomosis in tissues, veins or arteries in bad conditions and never would accept a clamping used in conventional anastomosis. This is achieved by a generally cylindrical shaped prosthesis with a flange orthogonally extending from its external side wall, in a point in the prosthesis length between its ends; the referred flange has openings distributed around its surface. The description of the usage method and specific construction of the prosthesis is presented in the drawings of the descriptive report of that request, as well as the document C19706197-2, Certificate of Addition of the first.

Although these anastomotic devices can be presented as suited to the purposes for which they were projected, they are not so suited for the purposes of the present invention, as described herein below.

### SUMMARY OF THE INVENTION

The present invention refers to variations of the currently known anastomotic devices, in order to allow side-to-side, end-to-end and end-to-side anastomosis without clamping and sutureless, or with quick clamping and sutureless, in organs with normal walls, where at least one vascular graft, or any other, is inserted in the lumen of the prosthesis and rotated by jacketing to cover part of the prosthesis, and it is fixed to the tubular member of the prosthesis, by a round point. The tubular member of the prosthesis, differently from the prior technique, besides having its intraluminal part angled in comparison to the flange, it can also have more than one intraluminal part, in form of Y, T, straight line, among other variations. Also, these intraluminal parts can be articulated, in fixed size, retractable or expansible (accordion type, made of any biocompatible material), with or without elastic memory, and can change the angle in relation to the flange and among them. This makes easier the introduction of the anastomotic set under the light of organs, reducing the size of the necessary incision and requiring a minimum mobilization of the organs walls in its habitual position, eliminating the need of dissection of the structures around it. The flange can be or not provided with openings and chamfers, allowing the sewing in the exterior of a tissue, vein, artery or tubular organ to eliminate the contact of foreign bodies inside the anastomosis. The prosthesis can also present sizes and varied shapes to accommodate simultaneously the sizes, number and varied types of grafts.

One objective of the present invention is to provide an anastomotic device with a tubular member that ramifies in more than one intraluminal part, and this intraluminal part is angled in relation to the flange, being fixed or articulable, it can change its position in any direction, allowing the insertion of more than one graft, of different types and gauges (for example, anastomotic trunk with autologs, homologs, heterologs grafts) in the same prosthesis.

Another objective of the present invention is to provide an anastomotic device that does not introduce any foreign body in contact with grafts at anastomosis site, allowing sutureless anastomosis. Another objective of the present invention is to provide an anastomotic device that allows anastomosis without clamping and hermetically closed.

Another objective of the present invention is to provide an anastomotic device which grafts do not kink, independently at which pressure they are submitted to and where they are positioned.

Other features and additional objectives of the present invention will become apparent from the following descriptions. These features will be described at sufficiently detailed levels to allow the technicians of the subject matter to implement the invention. Also, it is understood that other features can be used and structural changes can be made without leaving the scope of the invention. In the accompanying drawings, like reference numbers indicate identical or like parts throughout the several views.

Therefore, the following detailed description should not be taken as limiting, and the scope of the present invention is better defined by the appended claims.

### BRIEF DESCRIPTION OF THE DRAWINGS

The present invention may be understood more completely by reference to the following description and appended drawings, supported by examples, in which:

FIG. 1A illustrates an upper view of the first prosthesis embodiment with intraluminal part angled from 0 to 180 degrees in relation to the flange plan, in all directions, retractable or extensible (accordion type), and intraluminal parts with external grooves, to fixate everted grafts with external round points. The flange comprising holes, double holes and/or external handles where the wires pass to fixate the prosthesis to the organ wall, without the interposition handles to the prosthesis light. FIG. 1B illustrates the perspective view of the first prosthesis embodiment with intraluminal part angled in relation to the flange plan, from 0 to 180 degrees, in all directions, retractable or extensible (accordion type) or of fixed size, with fixed or variable position, with or without elastic memory, the flange comprising holes, double holes and/or external handles to pass the fixation points of the prosthesis to the organ wall. The flange can also be flexible, of tissue (Dacron, PTFE, Gor-tex type etc.) without holes or handles.

FIG. 2A illustrates an upper view of a second embodiment of the prosthesis with intraluminal part angled from 0 to 180 degrees in relation to flange plan, the flange comprising internal and external holes, double holes and/or external handles where the wires pass to fixate the prosthesis to organ and chamfers, where the wire will be placed after passing through external holes and will be tied-up, tying circumferentially the organ that received the prosthesis.

FIG. 2B illustrates an upper view of a second embodiment of the prosthesis with other chamfers configuration which is placed after the external holes of the flange.

FIG. 2C illustrates a perspective view of a second embodiment of the prosthesis, illustrated in FIG. 2A.

A FIG. 3A illustrates an upper view of an embodiment of the prosthesis with double intraluminal part, angled in relation to the flange plan, the intraluminal part joining in double or triple output, with a flange comprising internal and external holes, double holes and/or external handles where the wires will pass to fixate the prosthesis to the organ wall and chamfers, where one of the wires, used to fixate the prosthesis to the wall, will be laid down after passing through external holes, and will be tied-up, tying circumferentially the organ that received the prosthesis.

FIG. 3B illustrates a perspective view of prosthesis embodiment with double intraluminal part, as illustrated in FIG. 3A.

FIG. 4A illustrates an upper view of a second embodiment of the prosthesis with double intraluminal part angled in relation to the flange plan, an intraluminal part joining in double output with the flange only in direction of intraluminal parts, without existing by side, with an extension just to accommodate small holes.

FIG. 4B illustrates a perspective view of a second embodiment of the prosthesis with double intraluminal part and minimum flange, as illustrated in FIG. 4A.

FIG. 5A illustrates an upper view of an embodiment of the prosthesis with double intraluminal part in form of Y in relation to the flange plan, with intraluminal part in split in two congruent directions, an intraluminal part joining in double, triple or multiple output, extending over the flange, and with external grooves, the flange comprising internal and external holes, double holes and/or external handles where the wires pass to fixate the prosthesis to organ and chamfers, where the wire legs will be laid down after passing through external holes and will be tied-up, tying circumferentially the organ that received the prosthesis.

FIG. 5B illustrates a perspective view of a second embodiment of the prosthesis with intraluminal part in form of Y, and a single tubular member, over the flange, with external grooves, for circumferential fixation of grafts which will cover its exterior. FIG. 5C illustrates other perspective view of a prosthesis embodiment with intraluminal part in form of Y, flange, and single tubular member over the flange, as illustrated in FIG. 5A and 5B.

### DESCRIPTION OF THE PREFERRED EMBODIMENTS

With reference to the detailed description of drawings, in which like reference numbers indicate identical elements throughout the several views, the figures illustrate one embodiment of the present invention, in the form of a prosthesis for anastomosis with at least one intraluminal part angled in relation to the flange.

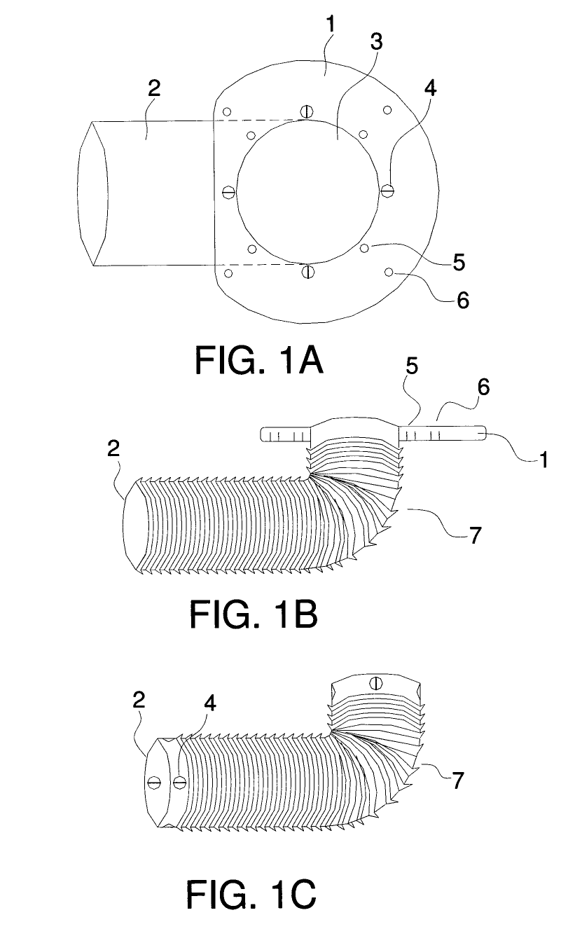

FIG. 1A illustrates a prosthesis with flange 1 with internal holes 5 and external holes 6, double or external holes, handles 4 with tubular member 2 angled in relation to the flange 1, extensible or retractable, movable for any direction, with external grooves 7 to anchor the grafts 9 fixed to it with round point.

FIG. 1B is a perspective view of prosthesis in FIG. 1A, where the angle and movable feature of the tubular member 2 are shown. The tubular member 2 is retractable or extensible and it can facilitate the eversion of grafts to cover it, as well as to its positioning and fixation in the light interior of the organ that received it. A technical embodiment for its use in a end-to-end anastomosis without clamping and sutureless could be described as follows: the graft 9 passes through the prosthesis light, it is everted to cover the intraluminal part 2 (for thick gauge prosthesis, the prosthesis itself will work as graft without the need of covering it, for example, prosthesis to substitute segments of aorta); then, the points for fixing prosthesis to the organ wall are applied, following the route: from top to bottom by internal holes 5, with anchored handles, in the middle, by double holes 4; it goes transfixing the organ wall and returns to external holes 6, being with its legs in parallel. All four points are applied in the same manner, two by two opposed and in parallel; the points are applied bordering the incision point, if it is a straight line, its length must be equal to half of external perimeter of prosthesis the tubular member. The free segment 9 of the graft is temporarily clamped and connected to prosthesis. The incision is made, digitally tamponed, the movable tubular member 2 in inserted and slightly accommodated to the light of the organ. The wires are tied-up. With another wire or wire legs directed to the intraluminal end of the tubular member 2, the tubular organ is circumferentially tied, over the prosthesis tubular member 2, being tied tight. With other wire or the opposed wire legs, the organ is also circumferentially tied under the prosthesis flange 1, turning on the liquid flow to that point.

With another end of graft 9, it is proceeded in the same manner, placing other prosthesis, resulting in a graft with two prosthesis, one in each end.

The graft 9 clamp is removed and the flow is processed by it. In each side, just one bag suture would be made in the upper surface of the wall or organs, and after the incision, the prosthesis would be introduced, circumferentially tying it (its tubular member) with a round point, fixing the organ to the prosthesis.

FIG. 1C illustrates a prosthesis that differs from prosthesis of FIG. 1A and 1B, because it does not have flange 1 and its holes, which are just double holes or small external handles 4, placed transversally on the intraluminal and external ends of the tubular member 2, next to its edges. Here, the wires can be previously fixed to double holes and/or small external handles 4, with the legs positioned out of the light of tubular member 2. The procedure for end-to-end anastomosis without clamping and sutureless can be the same as described above, although many other technical embodiments are possible.

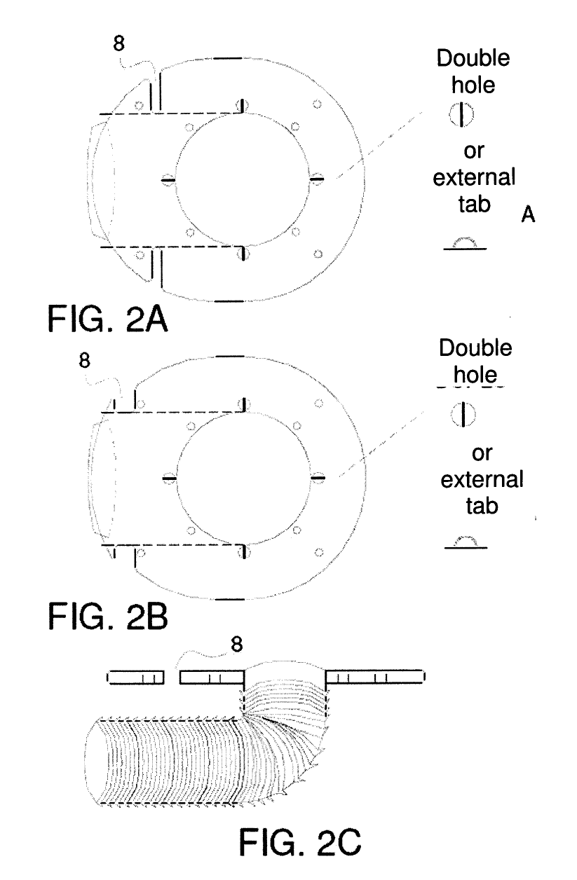

FIG. 2A illustrates a prosthesis that differs from prior art only by its chamfer 8 in the flange 1, before the external holes 6, to anchor the wire that will tie-up the organ to fixate it circumferentially and hermetically to the tubular member 2 of the prosthesis.

FIG. 2B illustrates a prosthesis which flange 1 differs from FIG. 2A, because it has a chamfer 8 to anchor the wire that will tie-up the organ, placed after the external hole 6, making the ligament as distal as possible over the tubular member 2, assuring that is will not slide forward, before the tubular member 2 end. So, the chamfer 8 assures that the flange 1 will have a maximum width equal to the gauge of the tubular member 2.

FIG. 2C is only a perspective view of the prosthesis of FIG. 2A, detailing the relation between the chamfer 8 and flange 1 and its localization in relation to tubular member 2.

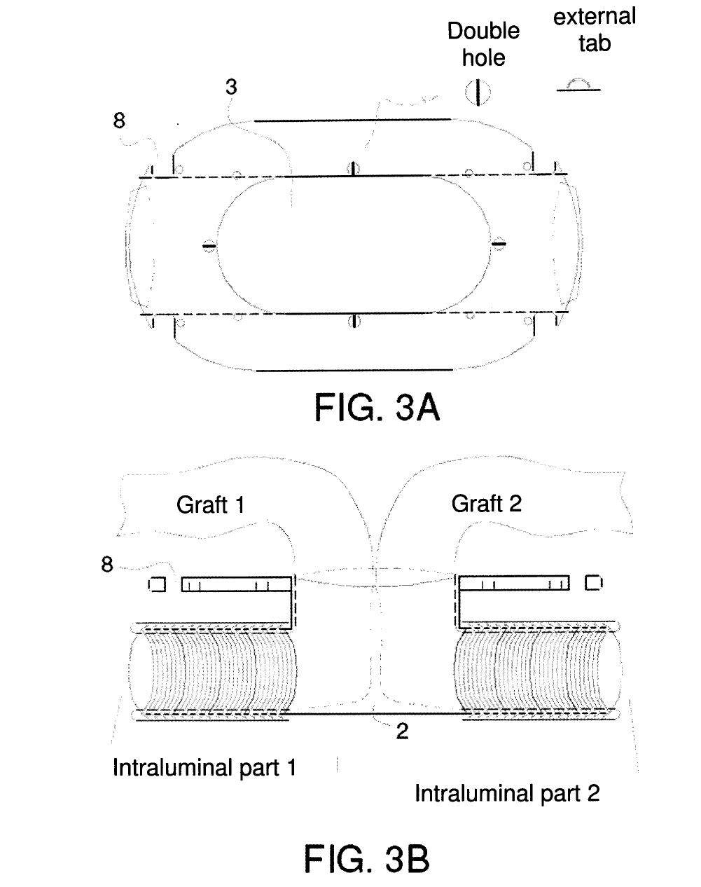

FIG. 3A illustrates an upper view of a prosthesis with two tubular members 2 that will be opposed in the final stage, and are fixed or movable (can vary the angle among them and/or between the flange), extensible or retractable, with external grooves 7 to anchor everted grafts 9, with single flange 1, elongated, with single output corresponding to the sum of the gauges of tubular members 2, with internal holes 5, external 6, double holes and/or small handles 4 and also chamfers 8 in its ends that serves to anchor the wire that will tie the organ over tubular members 2. Even the final position of tubular members 2 is diametrally opposed, the fact that they are movable (accordion type) can assume any direction and facilitate so much the insertions and positioning of light in the organ. The flange 1 can be made of tissue, flexible, without holes or handle and can be accommodate to the organ wall.

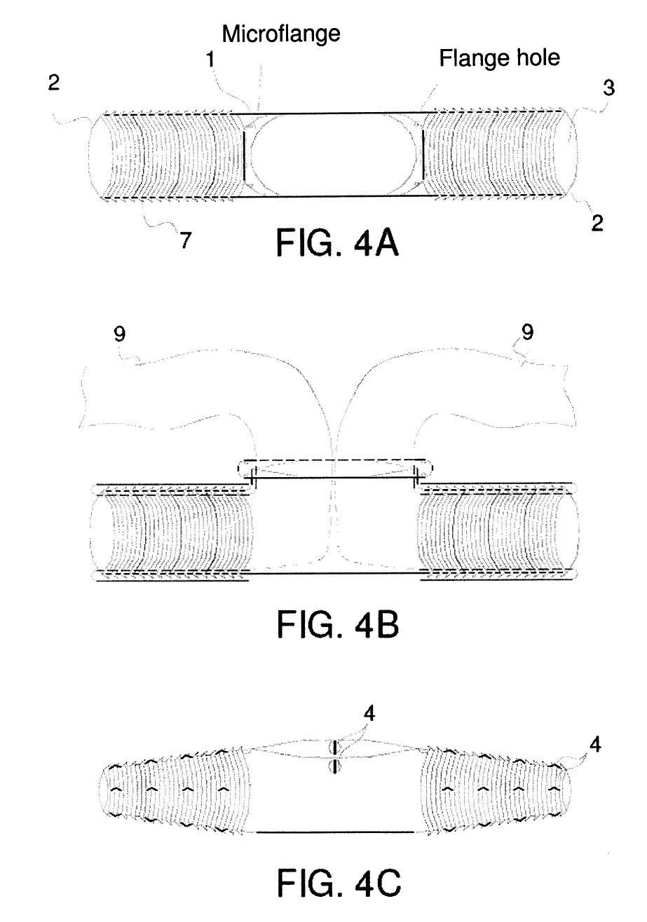

FIG. 4A illustrates a prosthesis that differs from the prior by its flange 1 that is only sufficient to comprise the double holes 4 and does not exist at the side. Also, the prosthesis can be rigid or flexible, extensible or retractable, with external grooves 7 to anchor the everted grafts 9.

FIG. 4B illustrates a prosthesis of FIG. 4A comprising two grafts 9, entering by the common opening, going to opposite sides, they are everted and cover the tubular member 2 that is fixed to them with an external round point. From the same source, the backward and forward flow will be assured by using this prosthesis. For example, the proximal and distal cables of a vessel with assurance to make perfectly and hermetically closed anastomosis. Also, the grafts do not present risk of kinking when emerging from prosthesis.

FIG. 4C illustrates a flangeless prosthesis, with double holes or small external handles 4 next to an edge of its common entrance, which shape is oval and the size is twice the biggest diameter of the tubular member, which in this prosthesis tapers from center to peripheral area, also comprising external grooves 7 to anchor grafts 9. These tubular members 2 can be rigid or flexible, retractable or extensible, movable or fixed, but always able to be cut when mounting the prosthesis grafts set to suit the gauge of proximal and distal cables in the organ where the anastomosis will be made. The tubular member 2 can have in its whole extension and circumferentially, equidistant, small external handles 4 where the grafts can be fixed, or where the wires that fix the prosthesis to proximal and distal cables of the organ can be applied. In this prosthesis, for example, grafts 9 of different gauges can be used passing through the same entry hole and directed to different places, however, having the same supply source, or being joined to prosthesis by its external segments.

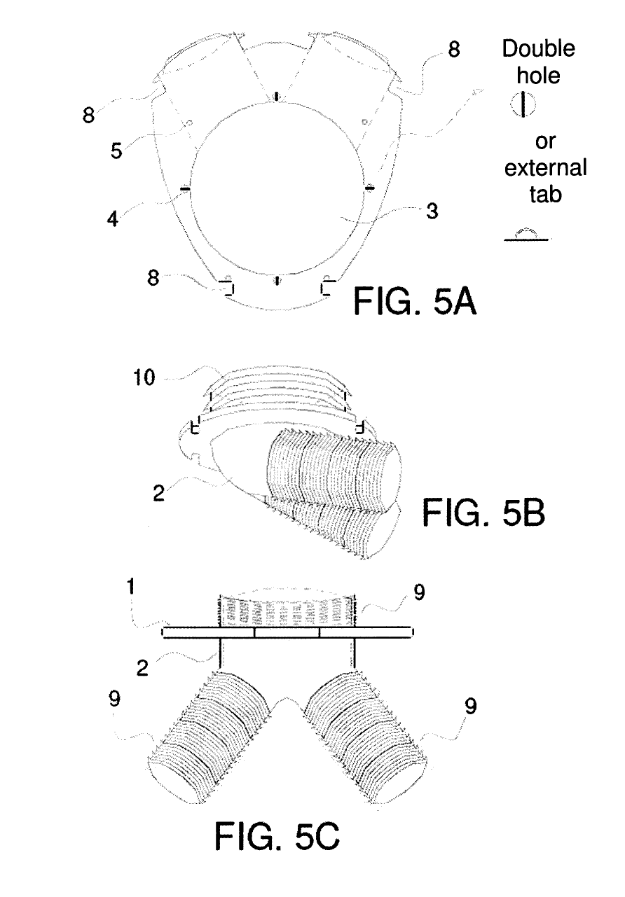

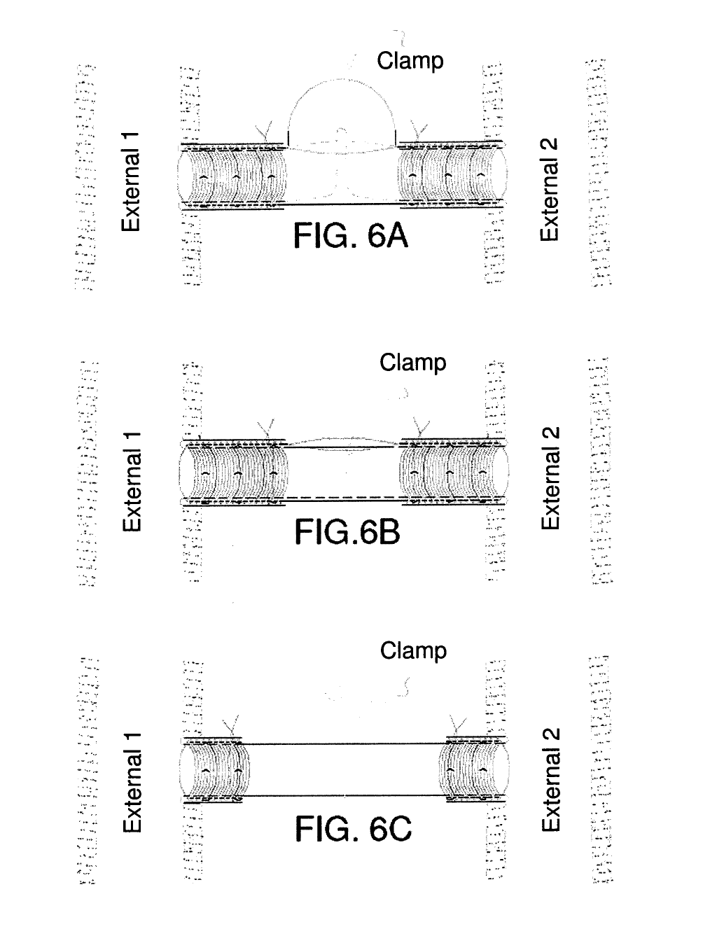

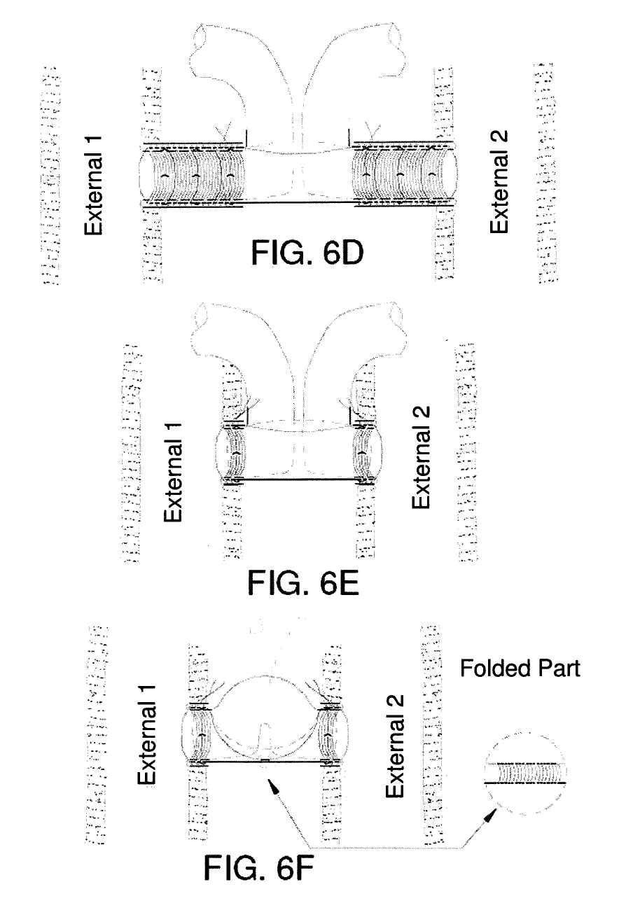

FIG. 5A illustrates a upper view of a prosthesis with double tubular member 2, intraluminal, fixed or movable (can have different extensions and gauges), rigid or flexible, extensible or retractable, which will assume the final position in the same direction in each corresponding parts of the flange 1, which shape allows, due to its individual chamfers 8, that each tubular members 2, at the final of prosthesis fixation to the organ wall where it was inserted, to be externally and separately tied-up, performing a perfect and hermetical fixation of tubular organs to them. The flange 1 also comprises other chamfers 8 opposed to its “legs” that serve to anchor the wire, if desired, will connect circumferentially proximal cable of the organ. The tubular members 2, before reaching the flange 1, join in a single opening, continuing upward over the flange 1, in a single header 10, big gauge (which gauge is equal to the sum of intraluminal parts gauges), which also comprise external grooves 7 to anchor grafts 9 or anastomotic trunks (formed by junction, by any method, of many grafts), that will cover the exterior, as represented in FIG. 5B and FIG. 5C. This prosthesis can come previously prepared by the industry, covered by homolog or heterolog grafts preserved or synthetic in the cases of prosthesis of thick gauge, avoiding the loss of time during the intraoperatory preparation.

These prosthesis allows end-to-side, end-to-end and side-to-side anastomosis without clamping and sutureless as demonstrated in side-to-side anastomosis illustrated in FIGS. 6A-6F.

1. Prosthesis for anastomosis comprising: at least one tubular member, a lumen and a flange extending from a side wall of the tubular member; the flange having a plurality of through openings distributed over the surface, wherein the tubular body is ramified at least in a intraluminal part (2) angled in relation to the flange (1).

2. Prosthesis, according to claim 1, wherein the intraluminal part (2) is angled from 0 to 180 degrees in relation to the flange (1).

3. Prosthesis, according to claim 1, wherein the prosthesis comprises two intraluminal parts (2) that extend in opposed directions.

4. Prosthesis, according to claim 1, wherein the prosthesis comprises two intraluminal parts (2) that extend in congruent directions.

5. Prosthesis, according to claim 1, wherein the flange (1) also comprises chamfers (8) in its perimeter.

6. Prosthesis, according to claim 1, wherein the through openings of flange are double holes or external handles (4).

7. Prosthesis, according to claim 6, wherein the double holes or external handles (4) are arranged equidistant throughout the extension of tubular members.

8. Prosthesis, according to claim 7, wherein the prosthesis does not have a flange (1).

9. Prosthesis for anastomosis comprising: at least one tubular member, a lumen and external grooves wherein the tubular member reduces the gauge from a center to a peripheral area or from an external part to an intraluminal part.

10. Prosthesis, according to claim 9, wherein the tubular member (2) or the intraluminal parts can be cut in size and gauge as desired.

11. Prosthesis, according to claim 10, wherein the prosthesis does not comprise a flange (1) and comprises double holes (4) or external handles that are equidistant, on an edge of the common lumen, passing through the the tubular member (2) thickness or through its exterior.
